(19) United States
(12) Patent Application Publication
(10) Pub. No.: US 2023/0347096 A1
(43) Pub. Date: Nov. 2, 2023

(54) POSITIVE PRESSURE BREATHING CIRCUIT

(71) Applicant: David John Love, Durban (ZA)

(72) Inventor: David John Love, Durban (ZA)

(21) Appl. No.: 18/041,381

(22) PCT Filed: Oct. 12, 2021
(86) PCT No.: PCT/NZ2021/050176

§ 371 Date: Feb. 10, 2023

(30) Foreign Application Priority Data
Dec. 8, 2020 (ZA) 2020/04960

(51) Int. Cl. A61M 16/12; A61M 16/20; A61M 16/10

(52) U.S. Cl. CPC A61M 16/122; A61M 16/209; A61M 16/1005; A61M 2205/3334

(57) ABSTRACT

The disclosure relates to a positive pressure breathing circuit and a method for ventilating a patient. The breathing circuit can be used in any type of pressurized breathing therapy including, for example, continuous positive air(way) pressure (CPAP) therapy and bilevel positive air pressure therapy where the inspiratory and expiratory pressures differ. The positive pressure breathing circuit comprises an inspiratory member including a distal portion connectable to a first gas and a proximal portion connectable to second gas wherein the inspiratory member is configured to store a volume of second gas. The inspiratory member further comprises a first non-return valve located proximally to the second gas entering the inspiratory member to inhibit the exhaled gases from entering the inspiratory member. The breathing circuit also comprises an expiratory member and second non-return valve to inhibit exhaled gases from re-entering the patient interface.

### CROSS-REFERENCE TO RELATED APPLICATIONS

This application claims priority from South African Patent Application No. 2020/04960, filed Aug. 12, 2020.

### FIELD

The present disclosure relates to a positive pressure breathing circuit and a method for ventilating a patient. The breathing circuit can be used in any type of pressurized breathing therapy including, for example, continuous positive air(way) pressure (CPAP) therapy and bilevel positive air pressure therapy where the inspiratory and expiratory pressures differ.

### BACKGROUND

Breathing circuits can help a patient to breath by opening up their airways and/or supplying specific breathing gases for a particular medicinal purpose. Breathing circuits may supply breathing gases at a flow rate that is higher than an average inspiratory flow rate to ensure there is no shortage of breathing gases. Specifically, in the case of constant positive airway pressure therapy, known as CPAP therapy, the flow supplied to the patient is usually higher than the peak inspiratory flow, rather than the average inspiratory flow.

This means that any breathing gases supplied during exhalation, or during a pause in breathing, may be wasted by being vented. There is therefore a need for a breathing circuit and a method that can reduce this wastage, or at least provide an alternative to existing breathing circuits.

### SUMMARY

An embodiment relates to a positive pressure breathing circuit for ventilating a patient, the breathing circuit including:
an inspiratory member with a gas passageway including a proximal portion that is connectable to a patient interface for supplying a breathing gas, and a distal portion that is connectable to a source of a pressurized first gas;
a pressure regulation device configurated to regulate pressure in the inspiratory member; and
an expiratory member configured to vent exhaled gases from the patient interface;
wherein the proximal portion of the inspiratory member is further connectable to a source of a pressurized second gas and the breathing circuit includes a first non-return valve that is arranged proximally to the second gas entering the inspiratory member, and the first non-return valve is configured to inhibit the exhaled gases from entering the inspiratory member, and the inspiratory member is configured so that a volume of the second gas can enter and be stored in the inspiratory member whilst the first gas can be supplied to the inspiratory member.

The first gas may be pressurized air. The first gas may be pressurized air enriched with oxygen.

In one example, the second gas may be pressurized oxygen gas.

In another example, the second gas may be a pressurized gas including one or any combination of: oxygen gas, heliox, or an anaesthetic gas. The anaesthetic gas could be nitrous oxide or a 50:50 mixture of nitrous oxide and oxygen gas.

Pressurized oxygen gas may be supplied from a liquified oxygen source, a bottled oxygen source or from an oxygen concentrator source.

During inhalation, the breathing gas the patient initially receives includes the pressurized second gas that is stored in the inspiratory member and the pressurized second gas entering the inspiratory member. When all of the stored oxygen gas has been inhaled or entered the patient interface, the breathing gas received by the patient includes the second gas entering the inspiratory member and the first gas in the inspiratory member. During exhalation the first non-return valve closes and the second gas entering the inspiratory member partially “backfills” the inspiratory member so as to replenish the stored (pressurized) second gas therein. The second gas is supplied to the inspiratory member at a higher pressure than the pressurized air supplied to the inspiratory member.

The inspiratory member may be sized to store a volume of the second gas that is supplied to the inspiratory member at a constant flow rate. An advantage of this feature is that there is no need to coordinate when the second gas is supplied to the patient, for instance, fluctuating the supply of the second gas during patient inhalation or exhalation.

The volume of the second gas, such as oxygen, that enters the inspiratory member during exhalation is equal to, or less than, a tidal volume of the patient, thereby minimising wastage of the second gas by venting pressurized air from the inspiratory member.

The gas passageway of the inspiratory member receives both the first and second gases during patient inhalation and exhalation.

The gas passageway of the inspiratory member has a constant internal volume. Although the gas passageway of the inspirator may be made of flexible material, the material is non-stretchable. That is to say, the internal volume of the passageway is relatively fixed in the sense that the gas passageway does not expand and does not inflate. For example, the passageway does not inflate during patient exhalation.

The inspiratory member can be an inspiratory tube, suitably an elongate inspiratory tube with an elongate gas passageway.

The inspiratory tube may have a length ranging from about 0.5 m to 2.5 m, or about a length ranging from 0.75 to 2.0 m, or a length ranging from about 1.5 to 1.8 m. The gas passageway may include a main passage of constant diameter, in which the diameter may range from about 18 to 25 mm, or the diameter is about 22 mm.

The inspiratory member may have an internal volume ranging from about 100 ml to 760 ml, for storing the second gas and some of the first gas. For example, the internal volume of the inspiratory member may be about 315, 350 or 500 ml.

In further examples, the internal volume may range from about 315 ml to 760 ml for adult patients, or range from about 400 to 600 ml. For pediatric patients, the internal volume may range from about 100 ml to 450 ml, or range from about 200 to 400 ml. For neonatal patients, the internal volume may range from about 50 to 200 ml, or range from about 100 to 150 ml.

The inspiratory member ideally has an internal volume that allows the pressurized oxygen gas that is stored in the inspiratory member to be inhaled by the patient in a single inhalation so that venting of the pressurized oxygen gas from the inspiratory member during exhalation can be avoided, thereby minimizing wastage of the pressurized oxygen gas.

The volume of the pressurized oxygen gas that may enter the inspiratory member during patient exhalation may range from about 50 to 90 percent by vol % of a tidal volume of a patient, or range from about 60 to 70 percent by vol % of a tidal volume of a patient.

The volume of the pressurized oxygen gas that may enter the inspiratory member during patient exhalation can equal an estimation of an alveoli volume of the patient.

The pressure of exhaled gases in the expiratory member may be greater than the pressure of the breathing gas in inspiratory member.

The expiratory member may include an expiratory tube having an elongate gas passageway.

The expiratory member has a second non-return device that can regulate the pressure at which gases are vented from the expiratory member.

The second non-return device inhibits gases exhaled from re-entering the patient interface via the expiratory member.

In one example, the second non-return device is a positive end expiratory pressure valve (PEEP valve). The positive end expiratory pressure valve of the expiratory member provides back pressure of the expired gases to the patient interface at a level that is operable to prevent oxygen gas leakage through the non-return valve of the inspiratory tube during exhalation.

In another example, the second non-return device is a bubbling bath in which the exhaled gases are required to exit the expiratory tube at a depth of liquid which determines the pressure at which gases are exhaled.

In one example, the pressure regulation device may include a first pressure relief valve configured to control the pressure of the first gas to the inspiratory member.

In another example, the pressure regulation device may include a second pressure relief valve configured to vent the first gas from the breathing circuit.

The pressure regulation device may include a further positive end expiratory pressure valve (further PEEP valve) on the distal portion of the inspiratory member.

The second pressure relief valve may be the further positive end expiratory pressure valve for the inspiratory member.

The pressure regulation device may include the inspiratory member including a further positive end expiratory pressure valve (further PEEP valve) on the distal portion of the inspiratory member.

The further positive end expiratory pressure valve may be configured to vent the pressurized first gas from the inspiratory tube during patient exhalation. In one example, the pressure in the inspiratory member is regulated by venting the pressurized first gas therefrom. In another example, the pressure in the inspiratory member may be regulated by adjusting the output of a gas flow generator, such as adjusting the speed of a fan of a gas flow generator. In yet another example, the pressure in the inspiratory member may be regulated by adjusting a gas flow control valve that supplies the first gas into the respiratory member.

The further positive end expiratory valve may vent pressurized air from the inspiratory member, in which more of the pressurized first gas is vented during patient expiration than during patient inhalation.

The positive end expiratory pressure valve of the expiratory member may have a pressure setting ranging from about 2.5 to 20.0 cmH₂O, or ranging from about 8.0 to 12.0 cmH₂O, or about 10.0 cmH₂O.

The further positive end expiratory pressure valve of the inspiratory member may have a pressure setting ranging from 0.5 to 1.0 cmH₂O less than the pressure setting of the positive end expiratory valve of the expiratory member. This reduces the likelihood of the breathing gas from being spontaneously discharged from the breathing circuit. More particularly, this arrangement inhibits the second gas from being vented from the breathing circuit by the positive end expiratory pressure valve of the expiratory member whilst the second gas is backfilling the inspiratory member.

The distal portion of the inspiratory member may have a first gas inlet adjacent to the further positive end expiratory pressure valve of the inspiratory tube, in which the first gas inlet is connectable to a source of the pressurized first gas.

The inspiratory member may be sufficiently long so that the stored second gas is inhibited from being discharged from the inspiratory member with the first gas via the further positive end expiratory pressure valve.

The positive end expiratory valve of the expiratory member may be operable to vent exhaled gases at a higher pressure than the pressure at which the first gas is vented from the inspiratory member.

The positive end expiratory valve and the further positive end expiratory valve may be operable to vent exhaled gases at a higher pressure from the expiratory member than the pressurized first gas from the inspiratory member respectively.

The positive end expiratory valve of the expiratory member may be a passive valve. For example, the positive end expiratory valve may have a fixed operating pressure or an operating pressure that can be manually adjusted. That is to say, the valve does not require active control measures or an actuator to continually monitor and adjust the operating pressure of the valve.

Similarly, the further positive end expiratory valve of the inspiratory member may be a passive valve. For example, the further positive end expiratory valve may have a fixed operating pressure or an operating pressure that can be manually adjusted. That is to say, the valve does not require active control measures or an actuator to continually monitor and adjust the operating pressure of the valve.

The positive end expiratory pressure valve of the expiratory member may have a higher pressure setting than a pressure setting of the further positive end expiratory pressure valve of the inspiratory member. In other words, there is a differential in the pressure settings that inhibits flow of the breathing gas from the inspiratory member to the expiratory member other than that caused by the patient.

The first non-return valve may be located on the proximal portion of the inspiratory member.

The first non-return valve may be located on the proximal portion of the inspiratory member, and be arranged proximal to where the second gas enters the inspiratory member. That is, the first non-return valve may be between the patient interface and where the second gas enters the inspiratory member.

The first non-return valve is located adjacent to the patient interface.

The proximal portion of the inspiratory member may have a second gas inlet upstream of the first non-return valve, at which the second gas inlet is connectable to the source of the pressurized second gas.

The breathing circuit may include a patient interface. The patient interface may be a sealed patient interface. For example, the patient interface includes either one or any combination of a full-face mask (also known as an oro-nasal mask), a sealed nasal cannula, a sealed oral mask, a sealed nasal mask, a nasal pillows interface, or a tracheostomy tube. The first non-return valve may be arranged on the patient interface.

The patient interface may have an inlet connection that connects to the inspiratory member, and an outlet connection that connects to the expiratory member.

The patient interface may have a coupling to which a Y-piece, is or can be connected, in which one leg of the Y-piece is an inlet connection that connects to the inspiratory member, and another leg is an outlet connection that connects to the expiratory member.

The positive end expiratory pressure valve of the expiratory member may be fitted directly to the outlet connection of the Y-piece.

The inspiratory member may be directly connected to the patient interface either with or without a Y-piece. That is to say, there are no intervening operations such as humidifiers, heat exchanges or other items that have the potential increase dead space in the breathing circuit between the inspiratory member and the patient interface.

The first gas may be continuously supplied to the inspiratory member.

The first gas may be supplied at a rate that is greater than or equal to the peak inspiratory flow rate of a patient. The peak inspiratory flow rate will be particular to individual patients.

The second gas may be supplied to the inspiratory member at a constant flow rate.

Another embodiment relates to a method of ventilating a patient, the method including steps of:
providing a positive pressure breathing circuit including:
   an inspiratory member with a gas passageway including a proximal portion that is connectable to the patient interface for supplying a breathing gas to the patient interface, the proximal portion including a first non-return valve that inhibits exhaled gases from entering the inspiratory member, and a distal portion configured to receive a pressurized first gas;
   a pressure regulation device configured to regulate pressure in the inspiratory member; and
   an expiratory member configured to vent exhaled gases from the patient interface;
supplying a pressurized second gas into the proximal portion of the inspiratory member, in which the second gas enters the inspiratory member at a location that is further along the inspiratory member from the patient interface than the non-return valve; and
supplying the pressurized first gas into the distal portion of the inspiratory member such that during patient exhalation, a volume of the pressurized second gas is configured to enter and be stored in the inspiratory member.

The step of supplying the pressurized first gas may be carried out continuously to the distal portion of the inspiratory member.

The step of supplying the pressurized first gas may be carried out at a rate that is greater than or equal to peak inspiratory flow rate of a patient.

The expiratory member may have a positive end expiratory pressure valve that is configured to vent the expired gases and inhibit the exhaled gases from re-entering the patient interface.

In one example, the pressure regulation device may include a first pressure relief valve configured to control the pressure of the first gas supplied to the inspiratory member, and the method include operating the first pressure relief valve.

In another example, the pressure regulation device may include a second pressure relief valve configured to vent the first gas from the breathing circuit, and the method may include operating the second pressure relief valve.

The pressure regulation device may include the inspiratory member including a further positive end expiratory pressure valve, and the method may include a step of setting the pressure at which the further positive end expiratory pressure valve vents excess first gas from the inspiratory member.

The method may include selecting a pressure setting of at least one of the positive end expiratory pressure valve of the expiratory member and the further positive end expiratory pressure valve of the inspiratory member, so that the pressure setting of the positive end expiratory pressure valve of the inspiratory member is lower than that of the expiratory member. The second pressure relief valve may be the further positive end expiratory pressure valve for the inspiratory member.

The method may include selecting a pressure setting of the positive end expiratory pressure valve of the expiratory member within a range from about 2.5 to 20.0 cmH₂O, or a range from about 8.0 to 12.0 cmH₂O, or about 10.0 cmH₂O.

The method may include selecting a pressure setting of the positive end expiratory pressure valve of the inspiratory member that ranges from about 0.5 to 1.0 cmH₂O less that the pressure setting of the positive end expiratory valve of the expiratory member.

The step of supplying the second gas may include controlling the flow rate of the second gas to the inspiratory member at a rate depending on the requirements of the patient. For instance, when the second gas is oxygen gas the flow rate will depend on the oxygen saturation concentration of the patient's blood.

The flow rate of the second gas may be controlled independently of any one or any combination of:
i) the tidal flow of the patient;
ii) changes in tidal flow of the patient; or
iii) a flow rate at which the pressurized air is supplied into the inspiratory member.

In other words, the flow rate of the second gas supplied to the inspiratory member can be determined so that the volume of the second gas stored in the inspiratory member tube during exhalation and supplied to the inspiratory member during inhalation will occupy the alveoli volume of the patient, meaning that enriched second gas need not be vented from the breathing circuit or drawn into dead space of the patient or of the breathing circuit. As a result, there will be less wastage of the second gas by reducing venting of the second gas from the breathing circuit without being inhaled, and by having a higher portion of the second gas in alveoli volume of a patient's lungs than in the dead space.

In the situation where the second gas includes enriched oxygen gas, controlling the flow rate of the oxygen gas supplied to the inspiratory member may be based on a level of oxygen saturation in the patient's blood.

During patient exhalation, the second gas entering the inspiratory member can flow backwards along the inspiratory member which acts as a constant pressure storage volume by displacing air out of the inspiratory member via the further positive end expiratory pressure valve of the inspiratory tube.

During patient inhalation, the breathing gas from the inspiratory member will initially be the second gas that had been stored in the inspiratory member and then the first gas. That is to say, during patient inhalation, the breathing gas the patient initially receives includes the second gas that was stored in the inspiratory member before the first gas.

When the first gas is air, supplying the air may be carried out in the range from about 2 to 120 l/min.

For example, in the case of an adult patient, the air may be supplied to the inspiratory member at a range from about 40 to 120 l/min, or range from about 50 to 70 l/min. In the case of pediatric patients, the air may be supplied to the inspiratory member at a range from about 3 to 50 l/min, or a range from about 4 to 40 l/min. In the case of neonatal patients, air may be supplied to the inspiratory member at a range from about 2 to 10 l/min, or at a range from about 3 to 6 l/min.

The inspiratory tube may have a length ranging from about 0.5 m to 2.5 m, or a length ranging from about 0.75 to 2.0 m, or a length ranging from about 1.5 to 1.8 m. The gas passageway may include a main passage of constant diameter, in which the diameter may range from about 18 to 25 mm, or a diameter about 22 mm.

The inspiratory member may have an internal volume ranging from about 100 ml to 760 ml, for storing the second gas and some of the first gas. For example, the internal volume of the inspiratory member may be about 315, 350 or 500 ml.

In further examples, the internal volume may range from about 315 ml to 760 ml for adult patients, or range from about 400 to 600 ml. For pediatric patients, the internal volume may range from about 100 ml to 450 ml, or range from about 200 to 400 ml. For neonatal patients, the internal volume may range from about 50 to 200 ml, or range from about 100 to 150 ml.

Another embodiment relates to a continuous positive air pressure breathing circuit for a patient, the breathing circuit including:
an inspirator that is connectable to the patient delivery device that supplies breathing gas to a patient;
a pressure regulation device configured to regulate pressure in the inspirator; and
an expirator configured to vent expired gases from the patient delivery device;
wherein the inspirator is connectable to a source of pressurized first gas and a source of pressurized second gas to provide the breathing gas, wherein the inspirator includes a first non-return device configured to inhibits exhaled gases in the patient delivery device from entering the inspirator, and the expirator is connectable to a second non-return device configured to inhibit expired gas from re-entering the patient delivery device via the expirator.

The inspirator may be an inspiratory tube.

The expirator may be an expired gas tube.

The first non-return device of the inspirator may be a non-return valve located adjacent to the patient delivery device.

The first non-return device of the inspirator may be located close to an inlet connection on the patient delivery device so that no expired gases can be discharged into the inspirator.

The first non-return device of the inspirator may be a non-return valve located between the patient delivery device and where the second gas enters the inspirator.

The second non-return device may be a positive end expiratory pressure valve that can be fitted to the expirator.

The positive end expiratory pressure valve may provide back pressure of expired gases and may be set to be sufficient to inhibit oxygen leakage through the first non-return means during exhalation.

In one example, the pressure regulation device may include a first pressure relief valve configured to control the pressure at which the first gas is supplied to the inspirator.

In another example, the pressure regulation device may include a second pressure relief valve configured to vent the first gas from the inspirator.

The pressure regulation device may include a further positive end expiratory pressure valve for venting the first gas from the breathing circuit. The second pressure relief valve may be the further positive end expiratory pressure valve for the inspirator.

The pressure regulation device may include a further positive end expiratory pressure valve for venting the first gas from the breathing circuit.

The further positive end expiratory pressure valve may vent the first gas from the breathing circuit without venting the second gas.

The inspirator may be connectable to the further positive end expiratory pressure valve that discharges excess pressurized air supplied to the inspirator upstream of the patient.

The positive end expiratory pressure valve of the expirator and the further positive end expiratory pressure valve of the inspirator each have a pressure setting and there is a differential in the pressure settings so as to prevent flow from the inspirator to the expirator other than that caused by the patient. For instance, the positive end expiratory pressure valve of the expirator has a higher pressure setting than the further positive end expiratory pressure valve of the inspirator.

The first gas may be supplied continuously to the inspirator. For example, at a constant flow rate.

The first gas may be supplied at a rate that is greater than or equal to peak inspiratory flow rate of a patient.

The first gas and the second gas are supplied concurrently to the inspiratory member.

An embodiment also relates to a method of operating the breath circuit described herein, wherein the method includes operating the breathing circuit at a positive pressure by maintaining an oversupply of the first gas into the inspirator.

The method may include supplying the first gas continuously to the inspirator.

The method may include supplying the first gas at a rate that is greater than or equal to peak inspiratory flow rate of a patient.

The method may include setting the pressure at a pressure which the further positive end expiratory pressure valve vents the first gas that is in excess from the inspirator.

When the expirator may have a positive end expiratory pressure valve for venting expired gas, and the inspirator has a further positive end expiratory pressure valve, the method may include selecting pressure settings of the positive end expiratory pressure valve of the expirator and the further positive end expiratory pressure valve of the inspirator so that there is differential between the pressure settings that will prevent net flow from the inspirator into the expirator other than that caused by the breathing of the patient.

The method may include controlling the flow of second gas to the inspirator at a fixed rate depending on the requirement of the patient.

The pressure regulation device may include a first pressure relief valve and the method include operating the pressure relief valve to control the pressure of the first gas supplied to the inspirator.

The pressure regulation device includes a second pressure relief valve configured to vent the first gas from the inspiratory member, and the method includes operating the second pressure relief valve to control the pressure of the inspiratory member

Another embodiment relates to a continuous positive air pressure ventilator comprising:
a) a source of a first gas;
b) a source of a second gas; wherein the first and second gas together comprise fresh gas;
c) a delivery device to deliver the fresh gas to a patient; the delivery device comprising:
   i) an inspirator for receiving fresh gas; and
   ii) a separate expirator for expired gas wherein the inspirator includes non-return means such that expired gas is prevented from substantially entering the inspirator means and the expirator includes non-return means such that expired gas is prevented from re-entering the delivery device via the expirator.

The first gas may be air. The second gas may be compressed oxygen. One or both of the first and second gases may pass through a humidifier before being delivered to the inspirator.

The non-return means on the inspirator means may be a non-return valve.

The non-return means on the expirator may be a positive end expiratory pressure valve.

The inspirator may further include a positive end expiratory pressure valve.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 is a schematic illustration of a positive pressure breathing circuit for ventilating a patient in which a pressurized first gas is supplemented using a pressurized second gas.

FIG. 2 is a schematic illustration of a positive pressure breathing circuit for ventilating a patient in which a first gas comprising pressurized air and a second gas comprising pressurized oxygen gas is used in the breathing circuit.

FIG. 3A is a schematic illustration of an inspiratory member of the positive pressure breathing circuit shown in FIGS. 1 and 2, in which the interface between the first gas, such as pressurized air, and the second gas, suitably pressurized oxygen gas, is a gas/gas interface.

FIG. 3B is a schematic illustration of an inspiratory member of the positive pressure breathing circuit shown in FIGS. 1 and 2, in which the boundary between the first gas and the second supplemental gas includes a plug device having a one-way valve.

FIG. 4 is a block diagram of a method steps for ventilating a patient using the breathing circuit shown in FIG. 1 or 2.

FIG. 5 is a graph illustrating a full breathing cycle of 6 seconds with an assumed tidal flow profile over an inhalation time that is one third of the cycle (2 seconds) and an exhalation time is two thirds of the breathing cycle (4 seconds). The graph illustrating the portion of the inhalation that is intended to occupy the alveolar volume of a patient's lungs. The minute ventilation being the tidal volume multiplied by the respiratory rate.

FIG. 6 is a graph illustrating the tidal flow during inhalation only, and shows individual flows, including flow of oxygen gas from and to the storage volume of the inspiratory member, flow of air into the inspiratory member, and the constant flow of the pressurized air into the respiratory member. A negative flow illustrates the flow of the secondary gas, such as supplemental oxygen gas into the inspiratory member during exhalation.

FIG. 7 is a graph illustrating the volume of the second gas, such as oxygen gas stored in the inspiratory member.

FIG. 8 is a graph illustrating the elevated concentration of second gas, such as oxygen gas in the patient's lungs. The graph is an average concentration of oxygen gas of the inspired gas expressed as volume fraction of oxygen, also known as FiO2.

FIG. 9 is a graph illustrating a full breathing cycle, as shown in FIG. 5, and details the flows during the breathing cycle that occur using the breathing circuit shown in FIGS. 1 and 2 according to a simulation.

### DETAILED DESCRIPTION

An embodiment will now be described in the following text which includes reference numerals that correspond to features illustrated in the accompanying drawings. To maintain clarity of the drawings, however, not all reference numerals are included in each figure of the drawings. Although certain examples are described herein, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described herein.

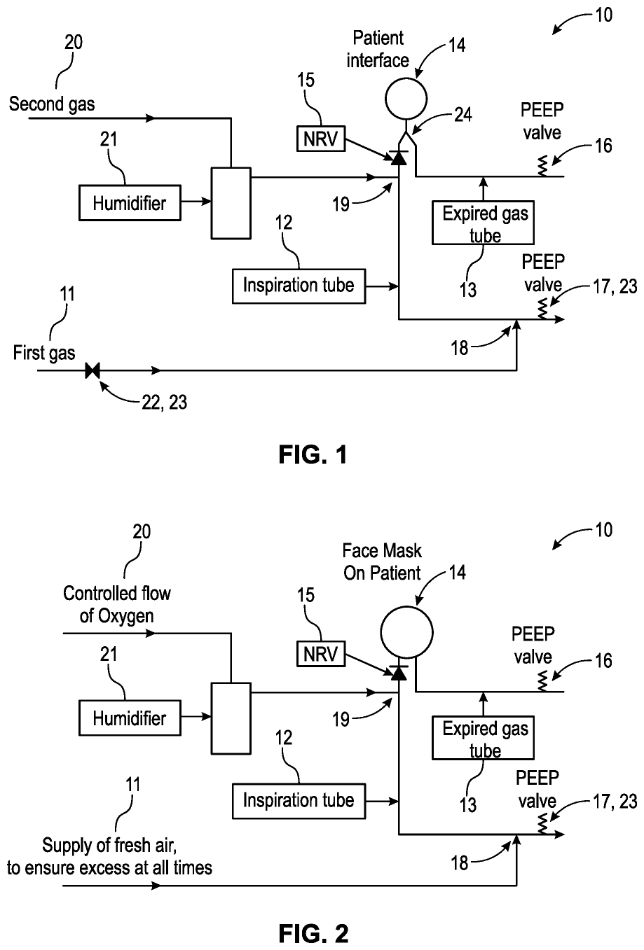

FIGS. 1 and 2 illustrates a positive pressure breathing circuit 10 for ventilating a patient. The breathing circuit can be used for different breathing therapies, including Continuous Positive Airway Pressure (CPAP) therapy and Bilevel Positive Air Pressure therapy where the inspiratory and expiratory pressures differ.

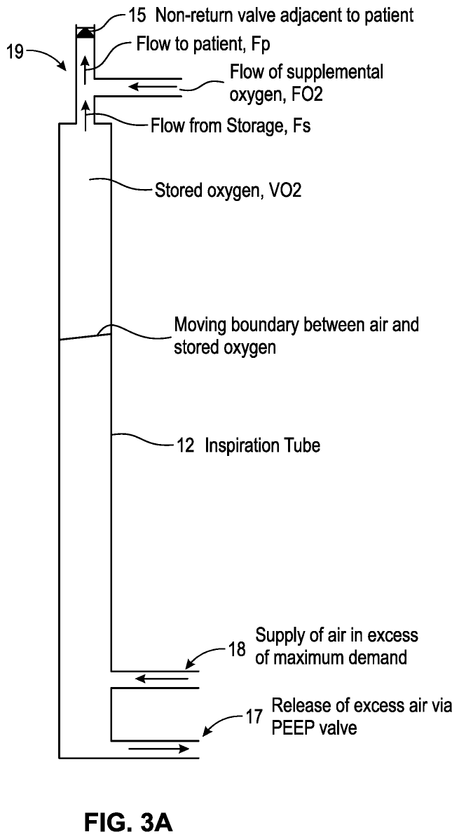

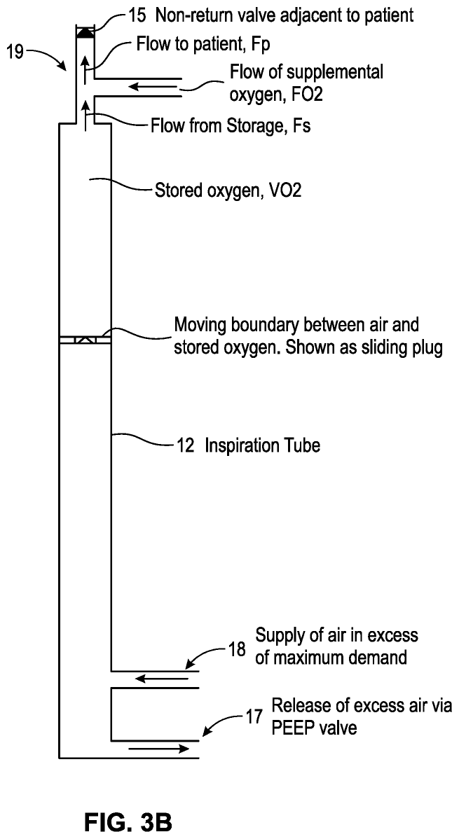

The breathing circuit 10 includes an inspiratory member 12 having a gas passageway, which in one example is also referred to as an inspiration tube. FIGS. 3A and 36 are schematic illustrations of the inspiratory member 12 which includes a proximal portion that is connectable to a sealed patient interface 14, which in one example is also referred to as a patient delivery device for supplying a breathing gas to the patient, and a distal portion includes a first gas inlet 18 that is connectable to a source of a pressurized first gas 11. The proximal portion of the inspiratory member includes a second gas inlet 19 that is connectable to a source of a pressurized second gas 20 and includes a first non-return valve 15 that is located upstream of the patient interface 14 and downstream of the second gas inlet 19 where the second gas 20 enters the inspiratory member 12. In other words, the first non-return valve 15 is located proximal to the second gas inlet 19. In another example, not illustrated, the first non-return valve 15 may be located on the patient interface 14.

The breathing circuit 12 also includes an expiratory member 13 that vents exhaled gases from the patient interface 14. In one example, the expiratory member 13 may be connected directly onto the patient interface 14. In another example, and depicted in FIGS. 1 and 2, the expiratory member 14 may include an expired gas tube 13. The expiratory member 13 includes a first non-return device in the form of a first positive end expiratory valve 16 (first PEEP valve) that regulates the pressure at which the gases are vented from the patient interface 14. The first PEEP valve 16 also inhibits exhaled gases from re-entering the patient interface 14 after having been exhaled.

The non-return valve 15 of the inspiratory member 12 inhibits the exhaled gases from entering the inspiratory member 12 and may be any suitable valve. Examples of suitable valves include a one-way flap valve, a biased valve that is biased into a closed position, or a diaphragm valve. The non-return valve 15 closes when the gas pressure downstream of the non-return valve 15 in the patient interface 14 is greater than the pressure in the gas passageway. The non-return valve 15 opens, suitably automatically, when the patient spontaneously inhales. The inspiratory member 12 and the expiratory member 13 including T-pieces or Y-pieces that supply the first and second gases 11, 20 to the gas passageway may be made of any suitable medical grade material.

The breathing circuit also includes a pressure regulating device 23 that regulates the pressure in the inspiratory member 12. For instance, the pressure regulation device may include a pressure relief valve 22 (see FIG. 1) for adjusting the flow and pressure of the first gas 11 entering the inspiratory member 12.

In the example shown in FIG. 2, pressure is regulated in the inspiratory member 12 by a second non-return device that vents the first gas 11 from the inspiratory member 12. The second non-return device may include a further positive end expiratory gas valve 17 (the further PEEP valve). It is intended that the pressure relief valve 22 and the second non-return valve, such as the further PEEP valve 17 be alternatives in the breathing circuit 10. It is also possibly, but less likely, the pressure relief valve 22 and the non-return valve such as the further PEEP valve 17, could be used in combination.

As illustrated in FIGS. 1, 2, 3A and 36, the further PEEP valve 17 is located at the end of the distal portion, or toward the end of the distal portion of the inspiratory member 12, and the first gas 11 enters the inspiratory member at a location that is proximal of the further PEEP valve 17. Although not illustrated in the figures, it is also possible that the further PEEP valve 17 could be located upstream of where the first gas 11 enters the inspiratory member 12.

The further PEEP valve 17 of the inspiratory member may have a pressure setting ranging from 0.5 to 1.0 cmH₂O less than the pressure setting of the PEEP valve 16 of the expiratory member. This inhibits the breathing gas from being spontaneously discharged from the breathing circuit 10. If this was not the case, the beathing gas could bypass the patient without being inhaled and the second gas could not accumulate in the breathing circuit during patient exhalation.

The further PEEP valve 17 may be disconnectable from the inspiratory member 12 and the first PEEP valve 16 may be disconnectable from the expiratory member 13 or the patient interface 14. The first PEEP valve 16 and the further PEEP valve 17 may be disconnected and reconnected as required using suitable couplings including 10 mm, 15 mm, 18 mm or 22 mm conduit couplings.

Depending on the operational requirements, either one or a combination of the first PEEP valve 16 or the further PEEP valve 17 can be any suitable valve, including a fixed value PEEP valve or an adjustable PEEP valve. The fixed value PEEP valve operates by a bias to remain closed until the upstream side of the PEEP valve is exposed to pressure that causes the valve to open until the pressure on the upstream side of the valve falls to or below the opening pressure. An adjustable PEEP valve has a bias that can be adjusted such that the pressure value at which the PEEP valve opens a can be adjusted as desired.

During patient inhalation and exhalation, the second gas 20 enters the proximal portion via the second gas inlet 19 at a constant rate, and the first gas 11 enters the distal portion via the first gas 11 inlet 18 at a constant rate. At the start of patient exhalation, or during a pause between inhalation ending and exhalation starting, the non-return valve 15 closes and the second gas 20 back fills the gas passageway of the inspiratory member 12. During patient exhalation, the second gas and the first gas 11 forms a gas/gas interface that moves along the gas passageway away from the second gas inlet 19 toward the distal portion, thereby storing a volume of the second gas 20 in the inspiratory member 12 during patient exhalation. The volume of the second gas 20, such as oxygen, that enters the inspiratory member 12 during exhalation is equal to, or less than, a tidal volume of the patient, thereby minimizing wastage of the second gas 20 by venting the first gas 11 from the inspiratory member 12 instead of the second gas 20. Ideally, the volume of the gas passageway of inspiratory member 12 is selected such that all of the second gas 20 that is stored in the inspiratory member 12 and the second gas 20 supplied into the inspiratory member 12 from the second gas inlet 19 during patient inhalation is equal to, or less than the alveolar volume of the patient.

Set out below in Table 1 is a list of exemplary internal volumes of an inspiratory member 12 having a 22 mm internal diameter. For useability and to ensure adequate internal volume is provided, the inspiratory member 12 has an internal diameter of 22 mm and a length in the ranging from 1.5 m to 1.8 m for adult patients.

TABLE 1

| Tube length (m) | Tube volume (ml) |
|---|---|
| 0.75 | 285 |
| 0.80 | 311 |
| 0.83 | 315 |
| 0.92 | 350 |
| 1.00 | 380 |
| 1.31 | 500 |
| 1.50 | 570 |
| 1.58 | 600 |
| 1.80 | 684 |
| 2.00 | 760 |

In the case of FIG. 3A, the second gas 20 and the first gas 11 have a gas/gas interface where some gas mixing can occur. In the case of FIG. 36, the interface between the second gas 20 and the first gas 11 is defined by a plug that slides along the gas passageway. The plug can have a one-way valve that allows the first gas 11 to flow through the plug when the plug reaches the constriction in the gas passageway or when the inspiration rate of the patient is greater than the flow rate of the second gas 20 entering the inspiration member 12.

The first gas 11 and the second gas 20 can be any suitable breathable gases. For example, the first gas 11 may be any breathable gas such as air, air enriched with oxygen, or any suitable anaesthetic gas. In the situation where the first gas 11 is air, air may be supplied to the inspiratory member 12 in the range of the 2 to 120 l/min depending on the patient. In the case of an adult patient, air may be supplied to the inspiratory member 12 in the range of the 40 to 120 l/min, or in the range of 50 to 70 l/min. In the case of pediatric patients, air may be supplied to the inspiratory member 12 in the range of the 3 to 50 l/min, or in the range of 4 to 40 l/min. In the case of neonatal patients, air may be supplied to the inspiratory member 12 in the range of the 2 to 10 l/min, or in the range of 3 to 6 l/min.

Similarly, the second gas 20 may be any breathable gas including any one or any combination of air enriched with oxygen, oxygen, helium, heliox, or any anaesthetic gas. The anaesthetic gas may be nitrous oxide or a 50:50 mixture of nitrous oxide and oxygen gas.

One of the benefits is that the first gas 11 is vented from the breathing circuit 10 with little venting, or no venting of the second gas 20. This enables better usage of the second gas 20, such as oxygen in the treatment of patients suffering from respiratory diseases during an outbreak, such as COVID-19. In other words, whilst oxygen efficiency can yield cost savings in treating patients in situations where the supply of oxygen is constrained an oxygen efficient system will allow more patients to be treated or to allow higher levels of oxygen enrichment to be provided to the same number of patients.

In one example, the sources 11, 20 of the first and second gases 11, 20 can be any suitable source, including pressure cylinders containing the required gases, or inwall hospital supply. In another example, flow generators including blowers can be arranged to draw the gas from a storage facility or from ambient air. For instance, the source 11 of the first gas 11 may be filtered air, ambient air, or ambient air that has been filtered. The source 11 of the air may be pressurized by a flow generator, and the source 20 of the second gas 20 may be compressed oxygen gas, such as a liquified oxygen source, a bottled oxygen source, or an oxygen concentrator source. In addition, the first and second gases 20 may optionally, be humidified prior to delivery to the patient. FIGS. 1 and 2 illustrate the second gas 20, for example oxygen, gas being humidified in a humidifier 21 to increase patient comfort.

In the situation where the breathing circuit 10 is used to provide supplemental oxygen gas as the second gas 20, the flow of oxygen gas can be adjusted based on patient response, for example the level of oxygen saturation in the patient's blood.

FIG. 2 illustrates an embodiment in which the patient interface 14 connects to the inspiratory member 12 and the expiratory member 13. FIG. 2 represents two examples, one of which being the patient interface 14, represented by the circle, having inlet outlet connections that are directly on the patient interface 14 for connecting to inspiratory member 12 and the expiratory member 13 respectively. The second example being the patient interface 14 has a Y-piece, which is not separately illustrated in FIG. 2, in which the inspiratory member 12 connects to one of the legs of the Y-piece, and the expiratory member 13 connects to the other leg of the Y-piece 24, and the third leg connects to an inlet/outlet port on the patient interface 14. FIG. 1 illustrates an embodiment in which the Y-piece connector 24 is part of the patient interface 14 and has been illustrated. One leg of the Y-piece connector 24 may be an inlet connection that connects to the inspiratory member 12, another leg of the connector 24 may be an outlet connection that connects to the expiratory member 13, and the third leg of the Y-piece 24 connects to a port of the patient interface 14. The Y-piece connector 24 may also be integrally formed with the patient interface 14 so as to not be disconnectable therefrom.

The non-return device of the expiratory member such as the first PEEP valve 16, and the non-return means of the inspiratory member such as the further PEEP value 17 can be used to regulate pressure of the inspiratory member 12 and indeed the breathing circuit 10. Thereby enabling a constant flow rate of the second gas 20 to be used, which is decoupled from regulating the pressure of the breathing circuit 10.

The following is a non-exhaustive list of some features of the breathing circuit illustrated in FIGS. 1, 2, 3A and 36.
i. The patient is fitted with a conventional respiration mask, suitably a sealed respiratory mask such as, a sealed full-face mask (also known as an oro-nasal mask), a sealed nasal cannula, a sealed oral mask, or a sealed nasal mask, or a nasal pillows interface.
ii. The mask has both an inlet connection and an outlet connection so that fresh gas and expired gas are handled separately (and an anti-asphyxiation valve as a safety device).
iii. A non-return (one way) valve 15 is fitted close to inlet connection on the mask so that no expired gasses can be discharged into the inspiration tubing.
iv. The expired gasses pass down a dedicated expired gas tube to be discharged via the first positive end-expiratory pressure (PEEP) valve 16 at the end of the end of the expired gas tube 13.
v. The first PEEP valve 16 at the end of the expired gas tube 13 also acts as a second non-return valve, ensuring that the patient is inhibited from inhaling any gas from the expired gas tube.
vi. Air is provided to the patient from an inspiration tube 12 that is connected to the non-return valve adjacent to the respiration mask.
vii. The supply of the first gas 11, such as air is added to the inspiration tube 12 a suitable distance away from the patient.
viii. The supply of the first gas 11, such as air can be from a source of compressed air, via a throttle valve (e.g. a standard medical air flow controller) or the source of the air could be provided by a dedicated “blower” that provides the required air flow at the CPAP ventilator pressure.
ix. The first gas 11, such as air can be filtered to ensure that it meets the necessary quality standards for ventilation.
x. The first gas 11, such as air can be supplied from a source of compressed air, and the air may pass through the humidifier 21 to increase patient comfort.
xi. A suitable positive gas pressure, such as air pressure can be maintained in the inspiration tube 12 by supplying more air than is required by the patient and discharging the excess air via the further PEEP valve 17 at the opposite end of the inspiration tube 12 to the distal portion.
xii. A controlled flow of second gas 20, such as supplemental oxygen, to assist the oxygen uptake of the patient is added to the inspiration tube 12 just prior to the non-return valve 15. This supply can be via a humidifier 21 to increase patient comfort.
xiii. The flow of second gas 20, such as oxygen is regulated directly to achieve the required patient response, rather than trying to adjust the oxygen to air ratio of the inspired gasses to achieve the required patient response.

### Practical Implementation, Operation and Control

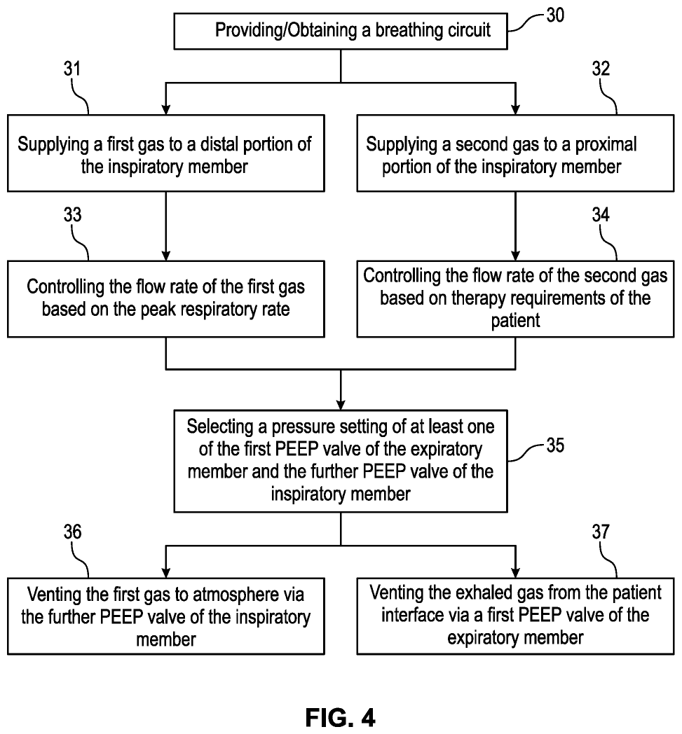

FIG. 4 is a block diagram illustrating the method steps of ventilating a patient using the breathing circuit shown in FIGS. 1 to 3B. The method includes providing or obtaining 30 the breathing circuit which may include connecting the inspiratory member 12 and the expiratory member 13 to the patient interface 14. In the instance where a patient interface 14 includes a Y-piece, the proximal portion of the inspiratory member 12 can be manually connected to one of the legs of the respective piece and the expiratory remember 13 can be manually connected to the other leg. Similarly, where the patient interface 14 includes an inlet connection and an outlet connection on a frame of the patient interface 14, the proximal portion of the inspiratory member 12 can be manually connected to the inlet connection and the expiratory member 13 can be manually connected to the outlet connection. A user may also connect the source 11 of the first gas 11 to the first gas inlet 18 at a distal portion of the inspiratory member 12, and connect the source 20 of the second gas 20 to the second gas inlet 19 at the proximal portion of the inspiratory member 12 for supplying 31, 32 of the first and second gases 11, 20.

Based on the assessment of the therapy requirements of the patient, flow rates of the first gas 11 and the second gas 20 to the inspiratory member 12 may be determined and controlled 33, 34 as shown in FIG. 4. Specifically, controlling 33 the flow rate of the first gas 11 is based on the peak respiratory requirement of the patient, and controlling 34 the flow rate of the second gas 20 is based on the therapy requirement of the patient. In the instance where supplemental oxygen therapy is required, the first gas 11, for example air, may be supplied at a rate, in the range of 40 to 120 l/min for adult patients, or 60 l/min. The flow rate of air supplied may exceed the peak respiratory flow rate requirement of the patient.

The second gas 20, for example oxygen, may be supplied at a flow rate based on the assessment of the oxygen saturation level of the patient's blood. For example, where the oxygen flow required is between 30 and 50% of the tidal volume, the oxygen flow may be controlled to range from 0.6 to 3.3 l/min.

Similarly, the user may select an inspiratory member 12 having the required internal volume to store the required amount of at least the second gas 20. For adult patients, where the respiratory member 12 has an internal diameter of 22 mm the respiratory member may, for example, have a length in the range of 1.5 to 1.8 m. The air flow rates, oxygen gas flow rates and length and internal diameter of the inspiratory member 12 can be selected by the user. Set up below in Table 2 are examples of flow rates and inspiratory member volumes for adult patients, pediatric patients and neonatal patients. As can be seen the flow rates and inspiratory member 12 volumes vary for each category of patient.

TABLE 2

| | | Peak | | Example inspiratory | | |
|---|---|---|---|---|---|---|
| | | inspiratory | | member internal | | |
| | Typical | flow | | dimensions | | Oxygen |
| | tidal | (while | | Dia × length | Air | flow |
| | volume | resting) | | (volume) | flow | 30%~50% |
| Adult | >300 ml, | 40~60 | l/min | 22 mm × 1.5 m | 60 l/min | 0.6 l/min~3.3 l/min |
| | typical 500 ml | | | (570 ml) | | |
| Paediatric | 50 ml < | 4~40 | l/min | 15 mm × 1.5 m | 40 l/min | 0.14 l/min~2.6 l/min |
| | x <300 ml | | | (265 ml) | | |
| Neonatal | <50 ml | 3~6 | l/min | 10 mm × 1.5 m | 6 l/min | 0.17 l/min~0.6 l/min |

(118 ml)

The method may include selecting 35 a pressure setting of at least one of the first PEEP valve 16 and the further PEEP valve 17 of the inspiratory member 12, so that the pressure setting of the further PEEP valve 17 of the inspiratory member 12 is lower than that of the first PEEP valve 16 of expiratory member 13. The pressure setting of the further PEEP valve 17 of the inspiratory member 12 ranges from 0.5 to 1.0 cmH₂O less than the pressure setting of the first PEEP valve 16 of the expiratory member 13.

The method may also include selecting 35 a pressure setting of the first PEEP valve 16 of the expiratory member 13 within a range from 2.5 to 20.0 cmH₂O, or ranging from 8.0 to 12.0 cmH₂O, or 10.0 cmH₂O.

As described above the pressure settings of the PEEP valves 16, 17 may be fixed or adjustable. In the case of fixed PEEP valves, each valve can be swapped out as required with a PEEP valve of the required pressure rating/setting.

Supplying the oxygen gas into the proximal portion of the inspiratory member 12, includes the oxygen gas entering the inspiratory member 12 on a distal side of the non-return valve 15. Furthermore the method may include supplying the pressurized air into the distal portion of the inspiratory member 12 during patient exhalation while a volume of the pressurized oxygen gas enters and is stored in the inspiratory member 12. As this occurs, the pressure in the inspiratory member is regulated via the further PEEP valve 17 of the inspiratory member 12 which vents 36 excess air from the inspiratory member 12. The oxygen gas may be supplied at a pressure greater than the pressure of the air so that the oxygen can backfill the inspiratory member 12. In other words, the second gas 20 is supplied at a pressure greater to the inspiratory member 12 than the first gas 11 so that the second gas 20 can backfill the inspiratory member 12. The first PEEP valve 16 of the expiratory member 13 vents exhaled gas 37.

One of the benefits of the breathing circuit 10 allows direct adjusting of the flow of the second gas 20, such as supplemental oxygen to the patient on the basis of the patient's response in terms of the level of oxygen saturation in the patient's blood. Conventionally measured with a pulse oximeter. This can be thought of as being equivalent to titrating the patient with oxygen to achieve the required response. An advantage provided by this method is that it avoids the excessive wastage of oxygen, or any second gas 20, in comparison to other high flow nasal cannular oxygen. The flow of the second gas 20 to the patient can be controlled irrespective of pressure and the total flow of the pressure therapy provided to the patient. The flow can be controlled independently of any one or any combination of:
i) the tidal flow of the patient;
ii) changes in tidal flow of the patient; or
iii) a flow rate at which the pressurized air is supplied into the inspiratory member.

The flow rate of the second gas 20 supplied to the inspiratory member 12 can be determined so that the volume of the second gas 20 stored in the inspiratory member 12 during exhalation and supplied to the inspiratory member 12 during inhalation will occupy the alveoli volume of the patient, meaning that enriched second gas 20 need not be vented from the breathing circuit 10 or drawn into dead space of the patient or dead space of the breathing circuit 10, if there is any. That is to say another operation benefit of the breathing circuit 10 is based on an understanding of alveolar “oxygen efficiency” which results from the design of the breathing circuit 10. This understanding can help avoid the oversupply of oxygen to a patient which provides no added benefit, avoiding oxygen wastage when the patient is experiencing close to 100% oxygen in the patient's lungs. Particularly if patient shows minimal response to increased oxygen concentration.

The breathing circuit 10 can achieve a high level of oxygen efficiency whilst reducing rebreathing of expired gasses. Some of the features of the breathing circuit 10 that contribute to these and other benefits are as follows.
i. The use of completely separate tubes, such as the inspiratory and expiratory members 12, 13 for the breathing gas and the expired gas reduces rebreathing of expired gases. It will be appreciated that rebreathing expired gases cannot be completely eliminated as a result gas mixing that happens between fresh breathing gas and the small quantity of exhaled gas remaining within the volume of the mask at the beginning of patient inhalation. However, the reduction in rebreathing in this way in turn eliminates the need to supply excess supplemental oxygen to be used to flush out expired gasses as a method of keeping the inspired level of CO2 at an acceptable level.
ii. To reduce rebreathing, the breathing circuit has the non-return valve 15 on the inlet to the patient interface 14, such as a face mask and the operation of the first PEEP valve 16 on the end of the expired gas tube 13 which operates as a non-return valve reducing expired gases from being re-inhaled.
iii. The positive air pressure in the breathing circuit 10 is maintained primarily by an oversupply of pressurized first gas 11, such as compressed air relative to maximum patient demand into the (distal) end of the inspiration tube 12 adjacent to the further PEEP valve.
iv. The excess air supplied into the inspiration tube 12 is discharged to atmosphere via the further PEEP valve 17 at the end of the inspiration tube 12. The cost of this “wasted” air is minimal and so this provides a cost-effective method of maintaining a constant pressure.
v. The operating pressure in the inspiration tube 12 is determined by the setting of the further PEEP valve 17 at the end of the inspiration tube 12.
vi. The settings of the first PEEP valve 16 and the further PEEP valve 17 are chosen to provide the appropriate Positive Air Pressure (PAP) for the patient. There may for example, be an appropriate (for example small) differential pressure between the pressure settings of the two PEEP valves 16, 17 to ensure that there will be no net flow from the inspiration tube 12 into the expired gas tube 13 other than that caused by the breathing of the patient. This arrangement can be conveniently and accurately set when using an appropriately designed bubbling system to replicate the function of conventional mechanical PEEP valves
vii. When the second gas 20 is oxygen gas, the supply of oxygen to the patient is set at a fixed rate depending on the requirements of the patient. One method of determining the patient's requirements is by measuring the oxygen saturation level in the patient's blood stream (using, for example, a pulse oximeter).
viii. During the exhalation period of the patient's breathing cycle the added oxygen will flow “backwards” along the inspiration tube 12 which acts as a constant pressure storage volume by displacing air in the tube 12 out via the further PEEP valve 17 at the end of the inspiration tube 12.
ix. The inspiration tube 12 is sufficiently long so that the stored oxygen does not mix with the added air at the end of the inspiration tube 12 and be discharged with the excess air via the PEEP valve 18.
x. In one example, the second gas 20, such as oxygen gas would displace the first gas 11, such as air in a plug flow fashion, but in reality there will be a degree of mixing between the oxygen and the air as the interface 14 between the oxygen and the air moves along the tube 12. The practical performance of the breathing circuit 10 can be estimated from a consideration of how the deviation from the plug flow situation will affect the operation of the breathing circuit 10.
xi. During the inhalation cycle, the initial gas drawn into the lungs from the inspiration tube 12 will be a combination of fresh oxygen from the supply tube combined with oxygen that had been stored in the inspiration tube 12 during the exhalation cycle.
xii. Thus, the first part of the inspiration cycle will inhale 100% oxygen, or close to it, depending on the extent of deviation from plug flow within the inspiration tube 12.
xiii. Towards the end of the inhalation cycle, assuming all the stored oxygen has been inhaled, the oxygen concentration of the inhaled gasses will drop to that determined by the fresh oxygen flow rate combined with the air flow necessary to meet the instantaneous inhalation demand (tidal flow).
xiv. This variation in oxygen concentration of the inhaled gas over the inhalation period can be particularly beneficial in terms of oxygen efficiency.
xv. The benefit results from the fact that the inhaled gas with the high oxygen concentration enters the alveoli where it is required whilst the inhaled gas with the low oxygen concentration towards the end of the cycle fills the “deadspace”, the bronchial tubes.
xvi. The dead space constitutes approximately 30% of the tidal volume and no oxygen is absorbed from this volume. Thus, added oxygen contained in the last 30% of the inhaled tidal volume is effectively wasted.
xvii. This breathing circuit 10 substantially reduces the wasted oxygen resulting from the dead space portion of the inhaled tidal volume with a minimum of extra complexity.
xviii. One advantageous feature of this breathing circuit 10 is its response to an increase in breathing rate (minute flow). The increase in flow will be supplied by an increase in fresh air being inspired by the patient (the oxygen flow having been set at a constant value). The increased air flow will help flush stored oxygen from the inspiration tube when there is mixing between stored oxygen and fresh air supply. The increased air flow will reduce the oxygen concentration of the inspired gas in the dead space, with the result that less of the added oxygen will be wasted due to being in this dead space and thus unavailable for absorption in the alveoli.

### Example

We have simulated the expected performance of the breathing circuit 10 described herein and illustrated in FIGS. 1 to 36 assuming that first gas 11 is air, and that the second gas 20 is supplemental oxygen gas. The simulation was based on a typical patient having a body mass of 70 kg and a tidal volume of 500 ml. Further details, including assumptions made, and settings of the breathing circuit are provided below in Table 3.

TABLE 3
Specification of Breathing & Ventilation

| | | |
|---|---|---|
| Patient mass | 70 | (kg) |
| Tidal Volume per mass | 7.14 | (ml/kg) |
| Tidal Volume | 500 | (ml) |
| Cycle Time | 6 | (sec) |
| Minute ventilation | 5.00 | (litres/min) |
| Respiratory Rate | 10 | (b/min) |
| Alveolar volume | 70.0% | (% tidal) |
| Alveolar volume | 350 | (ml) |
| Dead space volume | 150 | (ml) |
| Number of data points over cycle | 600 | |
| Data point step size (s) | 0.01 | (sec) |
| Supplemental oxygen flow | 2.5 | (litres/min) |

The simulation also included the assumption that the first and second gases 11, 20 obey ideal gases laws within the inspiration tube, that is the oxygen flowing into and out of the inspiration tube 12 exhibits “plug flow” behaviour and that there is no mixing between the oxygen and the air supplied into the inspiration tube. In the case of the embodiment shown in FIG. 3A, it will be appreciated that there will inevitably be some mixing, albeit minimal, at the interface between the oxygen gas and the air within the inspiration tube. The simulation assumes plug flow behaviour of the gases which is represented by the sliding plug between the first and second gases 11, 20 in FIG. 36. Although the principles and mathematics of deviations from “plug flow” (known as axially dispersed plug flow) are understood in theory, the extent of the deviations will be dependent on the specifics of the installation and its operating conditions and will need to be determined in practice. In most instances, these deviations from “plug flow” will affect the extent to which the breathing circuit achieves improved oxygen efficiency by reducing the quantity of supplemental oxygen that is present in the dead space of the inhaled volume. At lower supplemental oxygen flows, deviations from the assumed plug flow behaviour may have less impact, potentially resulting in little or no supplemental oxygen bypassing the patient. Thus, meaning the efficiency at which the supplement oxygen is used will approach 100%.

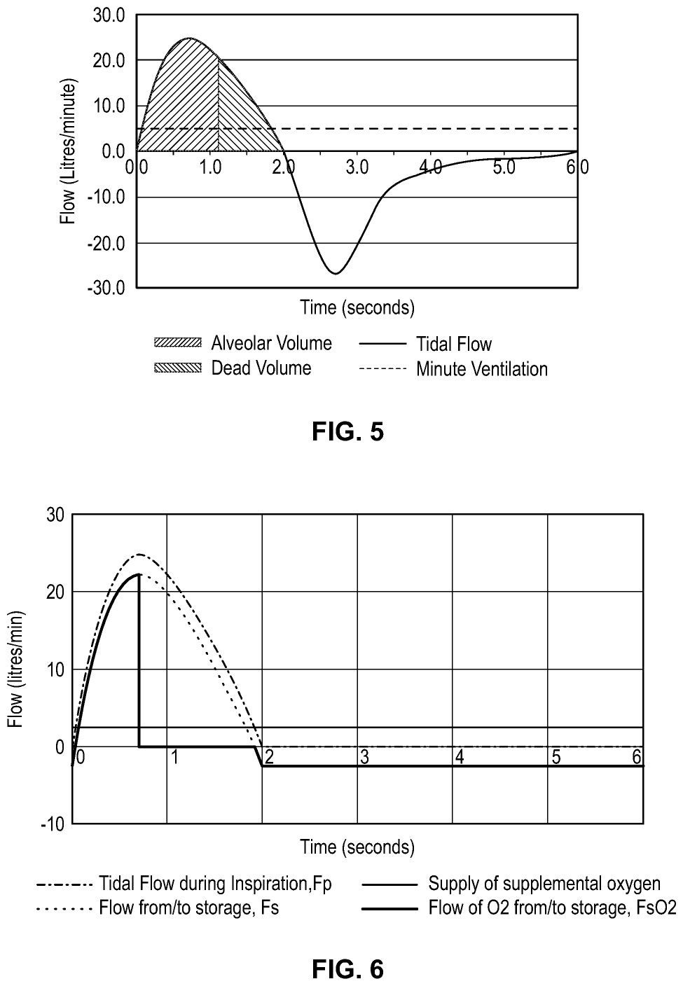

FIG. 5 illustrates the full breathing cycle used in the model which runs for 6 seconds with an assumed tidal flow trend that has an inhalation time that is one third of the cycle (2 seconds) whilst the exhalation time is two thirds of the breathing cycle (4 seconds). The inhalation flow is shown as positive while the exhalation flow is shown as negative. The volume of air inhaled is equal to the volume of air exhaled, but the shape of the inhalation and exhalation profiles are different, approximating conventional breathing patterns where there is a slow tailing off of flow during the exhalation portion of the cycle. FIG. 5 also shows a breakdown between the alveolar volume which is the portion of the inhalation that reaches the alveolar of the patient's lungs and therefore can be absorbed into the patient's lungs, and the dead space volume which does not participate in gas exchange.

FIG. 6 shows the tidal flow during inhalation which is indicated by the line comprising dots and dashes, and other gas flow in the breathing circuit. A negative flow from 2 seconds shows the flow of the supplemental oxygen gas into the inspiration tube that was then stored during the breathing cycle. As can be seen, the supplemental oxygen gas was stored in the inspiration tube of the breathing circuit when the inspirational tidal flow is less than the flow of supplemental oxygen.

During the initial portion of patient inhalation, the tidal flow is supplied by a combination of the supplemental oxygen flow entering the inspiration tube and stored oxygen drawn from the inspiration tube. Once the stored oxygen gas has been depleted, at approximately 0.7 seconds, the tidal flow was supplied by a combination of the supplemental oxygen flow and air drawn from the inspiration tube. The air being drawn from the inspiration tube is shown by the dotted line, and the supplemental oxygen flow is shown by the solid line.

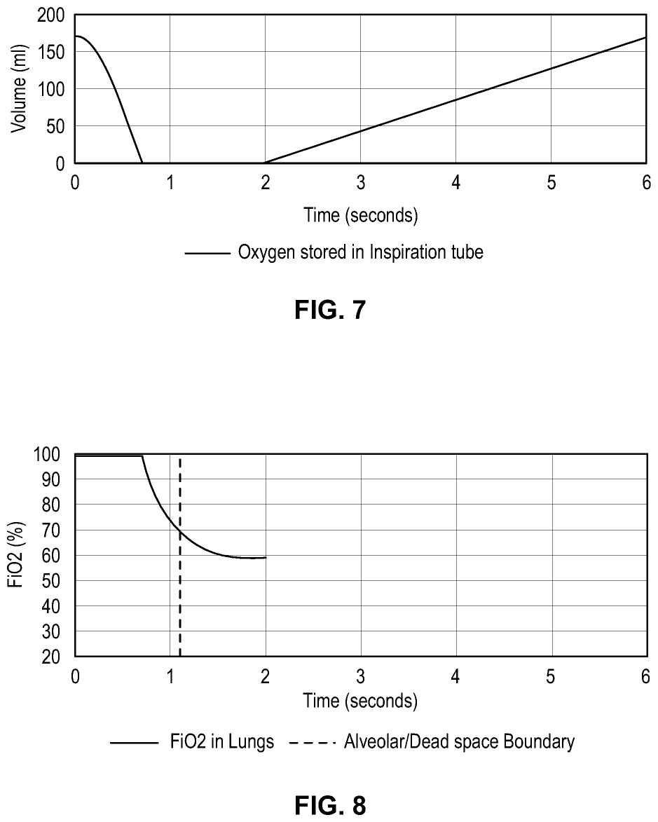

As can be seen in FIG. 7, the accumulation and storage of the supplemental oxygen in the inspiration tube decreases during the inhalation period, and is depleted at approximately 0.7 seconds, and remains at zero until the end of the patient inhalation. At the end of the patient inhalation, at approximately 2.0 second, the patient exhalation commences until 6.00 second. Between 2.0 and 6.0 seconds, the supplemental oxygen accumulates at a steady rate in the inspiration tube.

FIG. 8 plots the average concentration of oxygen of the inhaled gas, expressed as volume fraction of inhaled oxygen, referred to as FiO2, during patient inhalation. During the initial portion of patient inhalation the inspired gas will be 100% oxygen, which is supplied by a combination of supplemental oxygen flow into the inspiration tube and the stored oxygen. Once the stored oxygen has been depleted, the average oxygen concentration will begin to drop as a result of the inhalation of air together with the supplemental oxygen flow into the inspiration tube to meet the tidal flow demand of the patient. The stored oxygen depletes at approximately 0.7 seconds.

At the end of patient inhalation, the average oxygen concentration drops to the value that can be calculated directly from the patient's minute ventilation and the supplemental oxygen flow rate. Specifically, for the simulated conditions,
Minute ventilation=5.0 litres/min
Supplemental oxygen flow=2.5 litres/min
Thus, air flow=2.5 litres/min

Based on an oxygen concentration in air of 21% by volume, the average concentration of inhaled gas is 60.5% according to Equation 1:

(Air flow/Minute ventilation)*oxygen concentration of air+(Supplemental oxygen/Minute ventilation)*oxygen concentration of supplemental oxygen EQ 1.

2.5/5.0*21+2.5/5.0*100=60.5% EQ 1.

The vertical dashed line in FIG. 8 indicates the point at which the inhaled volume is equal to the alveolar volume of the patient's lungs, which was 350 ml for this simulation. This occurs at approximately 1.14 seconds. At this point the average FiO2 was 69.6%. This indicates an upper limit to the patient benefit in terms of effective FiO2, the average FiO2 within the alveolar volume that can be achieved by the breathing circuit for the conditions assumed for this simulation. Deviations from plug flow will reduce this benefit.

One of the outcomes of the simulation is that no supplemental oxygen will be wasted to atmosphere without being inspired by the patient. Moreover, we believe that substantial deviation from plug flow in the inspiratory tube can occur without causing any wastage of oxygen to atmosphere. More particularly, as the supplemental oxygen flow is increased towards the minute ventilation flow, this will raise the FiO2, the degree of deviation from plug flow (mixing between stored oxygen and air) will have an impact on the amount of oxygen gas that could be vented to the atmosphere. The extent of the impact will need to be determined in practice for the specific breathing circuit and its operating conditions.

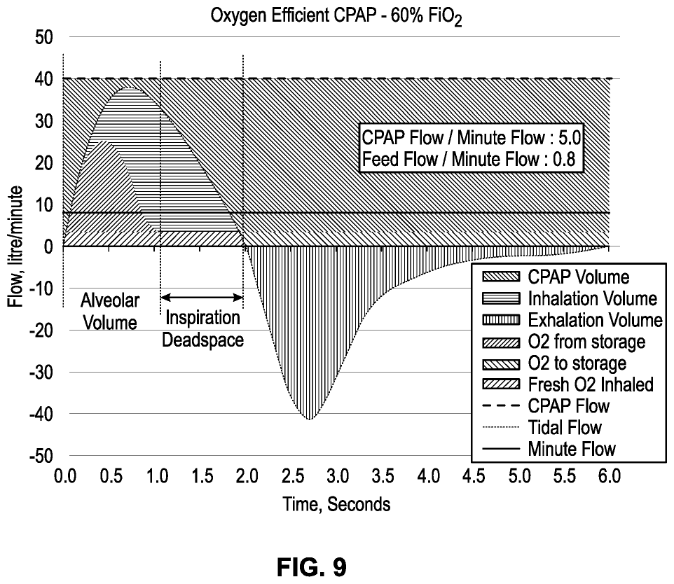

FIG. 9 is a graph showing the overall performance of the breathing circuit over the breathing cycle of the patient shown in FIG. 5. The breathing circuit was operating as a Highly Oxygen Efficient Continuous Positive Air(way) Pressure (HOE-CPAP) circuit. The graph illustrates that the oxygen inhaled by the patient is a combination of freshly supplied and previously stored oxygen. The simulation empirically includes air and oxygen mixing via axial dispersion in the inspiration limb whilst targeting an average FiO2 of 60%. The simulation shows that the axial dispersion of the stored oxygen in the inspiration tube 12 had no negative impact on the HOE-CPAP oxygen efficiency, because all the stored oxygen is inhaled into the Alveolar volume.

The CPAP flow rate was set at 40 l/min, being greater than the maximum respiration rate, and five times the minute ventilation rate of 8 l/min. The supplemental oxygen flow rate set a constant 2.5 l/min. As described above, tidal volume during patient inhalation comprised the stored oxygen and air until approximately 0.7 seconds, and from 0.7 second to 1.14 seconds fresh oxygen and air from the inspiratory tube was inhaled. From the start of patient inhalation until 1.14 seconds, the inhaled gases entered the alveolar volume at a FiO2 of approximately 69.6%. From 1.14 seconds to 2.0 seconds patient inhalation included the supplemental oxygen and air from the inspiration tube which entered the inspiration dead space. From 2.0 second to 6.0 seconds, patient exhalation occurred, during which, air was vented from the inspiration tube at a rate of CPAP flow rate plus the 2.5 l/min being rate at which the supplemental oxygen back filled the inspiration tube.

The simulation targeted a FiO2 of 60%. At this FiO2, no supplemental oxygen is wasted in the exhaled gases in the sense that all the stored oxygen that was inhaled preferentially entering the alveoli volume. Furthermore, there is now no rebreathing of CO₂ and so the exhalation profile and its duration have no impact on the rebreathing of CO₂.

In terms of the mechanisms that assisted in achieving this oxygen efficiency, there are three main aspects:
1. The breathing circuit 10 vents air to regulate pressure, whereas many other existing breathing circuits vent a combination of air and the supplemental oxygen. The excess air is vented upstream of the patient, rather than downstream as is the case with some existing breathing circuits. The breathing circuit 10 described herein has two PEEP valves 16, 17—one for excess air for venting the air from the inspiration tube 12, and the other for exhaled gases. The back pressure of the exhaled gas PEEP valve 16 is set to be (just) sufficient to reduce oxygen leakage through the non-return valve 15 during patient exhalation.
2. The breathing circuit 10 minimises the quantity of supplemental oxygen that is added to the “dead space” portion of the inhaled volume (the tidal volume). The gas that fills the “dead space” at the end of patent inhalation and is then emptied at the beginning of patient exhalation which is essentially unused gas, and any oxygen added into this portion of the inspired volume is essentially wasted or utilized in gas exchange as part of the breathing process.
3. The use of any combination of:
   a. a mask with a minimum air volume,
   b. two separate tubes 12, 13 connected to the mask, one for inhaled gas and the other for exhaled gas,
   c. the non-return valve 51 on the inspiration tube 12 close to the patient's mask,
   d. the PEEP valve 16 at the end of the expiration tube 13,
reduces and minimises the rebreathing of exhaled gasses. This in turn eliminates the need to consume oxygen enriched gas to flush out exhaled gas as a mechanism for reducing rebreathing, particularly of exhaled CO₂ to acceptable levels.

One of the operational features of the breathing circuit disclosed herein is that the gas from the dead space was vented with the exhaled gases, as opposed to some breathing circuits which focus on recovering and reusing the expired “dead space” gas. In this way the current proposal is substantially different.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or methods illustrated can be made without departing from the spirit of the disclosure. As will be recognized, certain embodiments described herein can be embodied within a form that does not provide all of the features and benefits set forth herein, as some features can be used or practiced separately from others.

### Definitions

In this specification, the following terms have the following meanings:
Tidal volume: Volume inhaled or exhaled with a single breath=7 ml/kg body weight (VT)
   Thus, for a standard 70 kg person,
   The tidal volume is 70*7=490 ml=0.49 litres
   Tidal volume is divided into alveolar and dead space volume.
Alveolar volume: Volume of a breath which enters the alveoli and partakes in gas exchange=approximately 70% of tidal volume (VA)
Dead space volume: Volume of a breath which fills the airways and does not participate in gas exchange=approximately 30% of tidal volume (VD) Dead space volume is relatively fixed, alveolar volume depends on tidal volume
Respiratory rate: Number of breaths per minute
Minute ventilation: Tidal volume×respiratory rate
Alveolar ventilation: Alveolar volume×respiratory rate
Dead space ventilation: Dead space volume×respiratory rate
Inspiratory flow rate: average flow in airway during inspiration approximately equal to:
   tidal volume×60/inspiratory time which, presuming:
      an I:E ratio of 1:2, and
      a respiratory rate of 10 (breaths per minute) gives a cycle time (period) for each breath of 6 seconds, which then means that Inspiratory time=2 secs and Expiratory time=4 secs
   Thus, for these conditions;
      Inspiratory flow rate=15 l/min
      (0.49 litres*60/2=14.7 l/min)
      Or, if the respiratory rate were to increase 20,
      Inspiratory flow rate=301/min
      (0.49 litres*60/1=29.4 l/min)
      As flow is not constant during inspiration the peak flow is high

1. A positive pressure breathing circuit for ventilating a patient, the breathing circuit including:
an inspiratory member with a gas passageway including a proximal portion that is connectable to a patient interface for supplying a breathing gas, and a distal portion that is connectable to a source of a pressurized first gas;
a pressure regulation device configured to regulate pressure in the inspiratory member; and
an expiratory member configured to vent exhaled gases from the patient interface;
wherein the proximal portion of the inspiratory member is further connectable to a source of a pressurized second gas and the breathing circuit includes a first non-return valve that is arranged proximally to the second gas entering the inspiratory member, and the first non-return valve is configured to inhibit the exhaled gases from entering the inspiratory member, and the inspiratory member is configured so that a volume of the second gas can enter and be stored in the inspiratory member whilst the first gas can be supplied to the inspiratory member.

2. The breathing circuit according to claim 1, wherein the volume of the pressurized second gas that enters the inspiratory member during exhalation is equal to, or less than, a tidal volume of the patient, thereby minimizing wastage of the pressurized second gas by venting the first gas from the inspiratory tube.

3. The breathing circuit according to any one of the preceding claims, wherein the gas passageway of the inspiratory member receives both the first and second gases during patient inhalation and exhalation.

4. The breathing circuit according to any one of preceding claims, wherein the passageway of the inspiratory member has a constant internal volume.

5. The breathing circuit according to any one of preceding claims, wherein the inspiratory member is sized to store a volume of the second gas that is supplied to the inspiratory member at a constant flow rate.

6. The breathing circuit according to any one of the preceding claims, wherein the inspiratory member includes an inspiratory tube that includes the gas passageway.

7. The breathing circuit according to any one of the preceding claims, wherein the expiratory member includes an expiratory tube.

8. The breathing circuit according to any one of the preceding claims, wherein the inspiratory member includes a tube defining the gas passageway, the tube including a length ranging from about 0.5 m to 2.5 m, or a length ranging from about 0.75 to 2.0 m for receiving the first and second gases.

9. The breathing circuit according to any one of the preceding claims, wherein the inspiratory member includes an internal volume ranging from about 100 ml to 760 ml.

10. The breathing circuit according to any one of the preceding claims, wherein the inspiratory member has an internal volume ranging from about 400 ml to 600 ml for adult patients, an internal volume of ranging from about 100 to 450 ml for pediatric patients, or an internal volume ranging from about 50 to 200 ml for neonatal patients.

11. The breathing circuit according to any one of the preceding claims, wherein the inspiratory member has an internal volume that allows the pressurized second gas that is stored in the inspiratory member to be inhaled by the patient in a single inhalation so that venting of the pressurized second gas from the inspiratory member during exhalation can be avoided, thereby minimizing wastage of the pressurized second gas.

12. The breathing circuit according to any one of the preceding claims, wherein the volume of the pressurized second gas that enters the inspiratory member during patient exhalation ranges from about 50 to 90 percent by vol % of a tidal volume of a patient, or from about 60 to 70 percent by vol % of a tidal volume of a patient.

13. The breathing circuit according to any one of the preceding claims, wherein the volume of the pressurized second gas that enters the inspiratory member during patient exhalation equals an estimation of an alveoli volume of the patient.

14. The breathing circuit according to any one of the preceding claims, wherein pressure of exhaled gases in the expiratory member is greater than the pressure of the breathing gas in inspiratory member.

15. The breathing circuit according to any one of the preceding claims, wherein the expiratory member has a second non-return device that can regulate the pressure at which gases are vented from the expiratory member.

16. The breathing circuit according to claim 15, wherein the second non-return device inhibits exhaled gases from re-entering the patient interface via the expiratory member.

17. The breathing circuit according to claim 15, wherein the second non-return device is a positive end expiratory pressure valve.

18. The breathing circuit according to claim 17, wherein the positive end expiratory valve of the expiratory member is a passive valve.

19. The breathing circuit according to claim 17 or claim 18, wherein the positive end expiratory valve has a fixed operating pressure or an operating pressure that can be manually adjusted.

20. The breathing circuit according to any one of claims 17 to 19, wherein the positive end expiratory pressure valve of the expiratory member provides back pressure of the expired gases to the patient interface at a level that is operable to inhibit oxygen gas leakage through the non-return valve of the inspiratory tube during exhalation.

21. The breathing circuit according to claim 15 or claim 16, wherein the second non-return device is a bubbling bath in which the exhaled gases are required to exit the expiratory tube at a depth of liquid which determines the pressure at which gases are exhaled.

22. The breathing circuit according to any one of the preceding claims, wherein the pressure regulation device includes a pressure relief valve configured to control the pressure of the first gas supplied to the inspiratory member.

23. The breathing circuit according to any one of claims 1 to 21, wherein the pressure regulation device includes a pressure relief valve configured to vent the first gas from the breathing circuit.

24. The breathing circuit according to any one of the preceding claims, wherein the pressure regulation device includes a further positive end expiratory pressure valve on the distal portion of the inspiratory member.

25. The breathing circuit according to claim 24, wherein the further positive end expiratory pressure valve is configured to vent the pressurized first gas from the inspiratory member during patient expiration.

26. The breathing circuit according to claim 25, wherein the further positive end expiratory valve vents the first gas from the inspiratory member, in which more of the pressurized first gas is vented during patient expiration than during patient inhalation.

27. The breathing circuit according to any one of claims 24 to 26, wherein the further positive end expiratory valve of the expiratory member is a passive valve.

28. The breathing circuit according to claim 27, wherein the further positive end expiratory valve has a fixed operating pressure or an operating pressure that can be manually adjusted.

29. The breathing circuit according to any one of claims 24 to 28, wherein the distal portion of the inspiratory member has a first gas inlet adjacent to the further positive end expiratory pressure valve of the inspiratory member, in which the first gas inlet is connectable to a source of the pressurized first gas.

30. The breathing circuit according to any one of claims 24 to 29, wherein the inspiratory member is sufficiently long so that the stored second gas is inhibited from being discharged from the inspiratory member with the first gas via the further positive end expiratory pressure valve.

31. The breathing circuit according to any one of claims 24 to 30, wherein the positive end expiratory valve of the expiratory member is operable to vent exhaled gases at a higher pressure than the pressure at which the first gas is vented from the inspiratory member during patient inhalation.

32. The breathing circuit according to any one of claims 24 to 31, wherein the positive end expiratory valve and the further positive end expiratory valve are operable to vent exhaled gases at a higher pressure from the expiratory member than the pressurized first gas from the inspiratory member respectively.

33. The breathing circuit according to any one of claims 24 to 32, wherein the positive end expiratory pressure valve of the expiratory member has a higher pressure setting than a pressure setting of the further positive end expiratory pressure valve of the inspiratory member.

34. The breathing circuit according to any one of claims 24 to 33, wherein the positive end expiratory pressure valve of the expiratory member may have a pressure setting ranging from about 2.5 to 20.0 cmH₂O, or ranging from about 8.0 to 12.0 cmH₂O, or about 10.0 cmH₂O.

35. The breathing circuit according to claim 34, wherein the positive end expiratory pressure valve of the inspiratory member may have a pressure setting ranging from about 0.5 to 1.0 cmH₂O less that the pressure setting of the positive end expiratory valve of the expiratory member.

36. The breathing circuit according to any one of the preceding claims, wherein the first non-return valve is located on the proximal portion of the inspiratory member.

37. The breathing circuit according to any one of the preceding claims, wherein the first non-return valve is located on the proximal portion of the inspiratory member, and is arranged proximal to where the second gas enters the inspiratory member.

38. The breathing circuit according to any one of the preceding claims, wherein the first non-return valve is located on the proximal portion of the inspiratory member is arranged adjacent to the patient interface.

39. The breathing circuit according to any one of the preceding claims, wherein the proximal portion of the inspiratory member has a second gas inlet upstream of the first non-return valve, at which the second gas inlet is connectable to the source of the second gas.

40. The breathing circuit according to any one of preceding claims, wherein the breathing circuit includes a patient interface.

41. The breathing circuit according to claim 40, wherein the patient interface has an inlet connection that connects to the inspiratory member, and an outlet connection that connects to the expiratory member.

42. The breathing circuit according to claim 40, wherein the patient interface has single coupling on the patient interface, and a Y-piece extending from the single coupling, in which one leg of the Y-piece is an inlet connection that connects to the inspiratory tube, and another leg is an outlet connection that connects to the expiratory tube.

43. The breathing circuit according to any one claims 40 to 42, wherein the patient interface includes either one or any combination of a sealed face mask, a sealed nasal cannula, a sealed oral mask, or a sealed nasal mask, a nasal pillows interface, or a tracheostomy tube.

44. The breathing circuit according to any one of the preceding claims, wherein the first gas is pressurized air.

45. The breathing circuit according to any one of the preceding claims, wherein the first gas is continuously supplied to the inspiratory member.

46. The breathing circuit according to any one of the preceding claims, wherein the first gas is supplied at a rate that is greater than or equal to peak inspiratory flow rate of a patient.

47. The breathing circuit according to any one of the preceding claims, wherein the second gas may be supplied to the inspiratory member at a constant flow rate.

48. The breathing circuit according to any one of the preceding claims, wherein the second gas is pressurized oxygen gas.

49. The breathing circuit according to any one of the preceding claims, wherein the second gas is pressurized one or any combination of: air enriched with oxygen, oxygen gas, heliox, an anaesthetic gas, or nitrous oxide.

50. A method of ventilating a patient, the method including steps of:
providing a positive pressure breathing circuit including:
an inspiratory member with a gas passageway including a proximal portion that is connectable to the patient interface for supplying a breathing gas to the patient interface, the proximal portion including a first non-return valve that inhibits exhaled gases from entering the inspiratory member, and a distal portion configured to receives a pressurized first gas;
a pressure regulation device configured to regulate pressure in the inspiratory member; and
an expiratory member configured to vent exhaled gases from the patient interface;
supplying a pressurized second gas into the proximal portion of the inspiratory member, in which the second gas enters the inspiratory member at a location that is further along the inspiratory member from the patient interface than the non-return valve; and
supplying the pressurized first gas into the distal portion of the inspiratory member such that during patient exhalation, a volume of the pressurized second gas is configured to enter and be stored in the inspiratory member.

51. The method according to claim 50, wherein the step of supplying pressurized first gas is carried out continuously to the distal portion of the inspiratory member.

52. The method according to claim 50 or 51, wherein the step of supplying pressurized first gas is carried out at a rate that is greater than or equal to peak inspiratory flow rate of a patient.

53. The method according to any one of claims 50 to 52, wherein the pressure regulation device includes a first pressure relief valve configured to control the pressure of the first gas supplied to the inspiratory member, and the method includes operating the first pressure relief valve.

54. The method according to any one of claims 50 to 52, wherein the pressure regulation device includes a second pressure relief valve configured to vent the first gas from the inspiratory member, and the method includes operating the second pressure relief valve.

55. The method according to any one of claims 50 to 54, wherein the expiratory member has a positive end expiratory pressure valve that is configured to vent the expires gases and inhibit the exhaled gases from re-entering the patient interface.

56. The method according to any one of claims 50 to 55, wherein the pressure regulation device includes the inspiratory member including a further positive end expiratory pressure valve, and the method includes a step of setting the pressure at which the further positive end expiratory pressure valve vents the first gas that is in excess from the inspiratory member.

57. The method according to claim 56 when appended to claim 55, wherein the method includes selecting pressure settings of the positive end expiratory pressure valve of the expiratory member to a higher setting than the pressure setting of the further positive end expiratory pressure valve of the inspiratory member.

58. The method according to claim 56 or 57, wherein the method includes selecting a pressure setting of the positive end expiratory pressure valve of the expiratory member within a range from about 2.5-20.0 cmH₂O, or ranging from about 8.0 to 12.0 cmH₂O, or about 10.0 cmH₂O.

59. The method according to claim 58, wherein the method includes selecting a pressure setting of the positive end expiratory pressure valve of the inspiratory member that ranges from about 0.5 to 1.0 cmH₂O less that the pressure setting of the positive end expiratory valve of the expiratory member.

60. The method according to any one of claims 50 to 59, wherein the step of supplying the second gas includes controlling the flow rate of second gas to the inspiratory member at a rate depending on the requirement of the patient.

61. The method according to claim 60, wherein controlling the flow rate of the second gas is controlled independently of any one or any combination of:
i) the tidal flow of the patient;
ii) changes in tidal flow of the patient; or
iii) a flow rate at which the first gas is supplied into the inspiratory member.

62. The method according to claim 60 or 61, wherein the second gas includes enriched oxygen gas or oxygen gas, and controlling the flow rate of the enriched oxygen gas or the oxygen gas supplied to the inspiratory member is based on level of oxygen saturation in the patient's blood.

63. The method according to any one of claims 50 to 62, wherein during patient exhalation, the second gas entering the inspiratory member will flow backwards along the inspiratory member which acts as a constant pressure storage volume by displacing the first gas out of the inspiratory member via the further positive end expiratory pressure valve of the inspiratory tube.

64. The method according to any one of claims 50 to 63, wherein during the patient inhalation, the breathing gas from the inspiratory member will initially be the second gas that had been stored in the inspiratory member and then the first gas.

65. The method according to any one of claims 50 to 64, wherein the first gas is pressurized air.

66. The method according to claim 65, wherein the air is supplied at a flow rate in the range from about 2 to 120 l/min.

67. The method according to claim 65, wherein the air is supplied to the inspiratory member at flow rate from about 40 to 120 l/min, or at range from about 50 to 70 l/min for an adult patient.

68. The method according to claim 65, wherein the air is supplied to the inspiratory member at a flow rate from about 3 to 50 l/min, or at a range from 4 to 40 l/min for pediatric patients.

69. The method according to claim 65, wherein the air is supplied to the inspiratory member at a flow rate from about 2 to 10 l/min, or at a range from about 3 to 6 l/min for neonatal patients.

70. The method according to any one of claims 50 to 69, wherein the inspiratory member has a tube defining the gas passageway, the tube having a length ranging from about 0.5 m to 2.5 m, or a length ranging from about 0.75 to 2.0 m for receiving the first and second gases.

71. The method according to any one of claims 50 to 70, wherein the inspiratory member has a tube defining the gas passageway, the tube having a length ranging from about 0.5 m to 2.5 m, or a length ranging from about 0.75 to 2.0 m for receiving the first and second gases.

72. The method according to any one of claims 50 to 71, wherein the inspiratory member has an internal volume ranging from about 100 ml to 760 ml.

73. The method according to any one of claims 50 to 72, wherein the inspiratory member has an internal volume ranging from about 400 ml to 600 ml for adult patients, an internal volume of ranging from about 100 to 450 ml for pediatric patients, or an internal volume ranging from about 50 to 200 ml for neonatal patients.

74. The method according to any one of claims 50 to 73, wherein the second gas is pressurized one or any combination of: oxygen gas, heliox, an anaesthetic gas, or nitrous oxide

75. A continuous positive air pressure breathing circuit for a patient, the breathing circuit including:
an inspirator that is connectable to the patient delivery device that supplies breathing gas to a patient;
a pressure regulation device configured to regulate pressure in the inspiratory member; and
an expirator configured to vents expired gases from the patient delivery device;
wherein the inspirator is connectable to a source of pressurized first gas and a source of pressurized second gas to provide the breathing gas, wherein the inspirator includes a first non-return device configured to inhibit exhaled gases in the patient delivery device from entering the inspirator, and the expirator is connectable to a second non-return device configured to inhibit expired gas from re-entering the patient delivery device via the expirator.

76. The breathing circuit according to claim 75, wherein the inspirator is an inspiratory tube.

77. The breathing circuit according to any one of claim 75 or 76, wherein the expirator is an expired gas tube.

78. The breathing circuit according to any one of claims 75 to 77, wherein the first non-return device of the inspirator is a non-return valve located adjacent to the patient delivery device.

79. The breathing circuit according to any one of claims 75 to 78, wherein the first non-return device of the inspirator is located close to an inlet connection on the patient delivery device so that little or no expired gases can be discharged into the inspirator.

80. The breathing circuit according to any one of claims 75 to 79, wherein the second non-return device is a positive end expiratory pressure valve that can be fitted to the expirator.

81. The breathing circuit according to claim 80, wherein the positive end expiratory pressure valve provides back pressure of expired gases and is set to be sufficient to prevent leakage of the second gas through the first non-return device during exhalation.

82. The breathing circuit according to any one of claims 75 to 81, wherein the pressure regulation device includes a first pressure relief valve configured to control the pressure of the first gas supplied to the inspirator.

83. The breathing circuit according to any one of claims 75 to 81, wherein the pressure regulation device includes a second pressure relief valve configured to vent the first gas from the inspirator.

84. The breathing circuit according to any one of claims 75 to 83, wherein the pressure regulation device includes the inspirator being connectable to a further positive end expiratory pressure valve that discharges the first gas that is supplied in excess to the inspirator upstream of the patient.

85. The breathing circuit according to claim 84 when appended to claim 83, wherein the positive end expiratory pressure valve of the expirator and the further positive end expiratory pressure valve of the inspirator each have a pressure setting and there is a differential in the pressure settings so as to inhibit flow from the inspirator to the expirator other than that caused by the patient.

86. The breathing circuit according to claim 83 or 84, wherein the inspirator has a first gas inlet toward an end of the inspirator adjacent to the further positive end expiratory pressure valve of the inspirator, the first gas inlet being connectable to the source of the pressurized first gas.

87. The breathing circuit according to any one of claims 75 to 86, wherein the inspirator has a second gas supply inlet that is located upstream of the non-return means of the inspirator that supplies the second gas at pressure into the inspirator, and the second gas supplied into the inspirator flows backfills along the inspirator which acts as a constant pressure storage volume by displacing first gas during exhalation.

88. The breathing circuit according to any one of claims 75 to 87, wherein the inspirator receives both the first and second gases during patient inhalation and exhalation.

89. The breathing circuit according to any one of claims 75 to 88, wherein the inspiratory member has a constant internal volume.

90. The breathing circuit according to any one of claims 75 to 89, wherein the inspiratory member has an internal volume ranging from 100 ml to 760 ml, and suitably from 315 ml to 670 ml for adult patients, and suitably the internal volume ranges from 100 ml to 450 ml for pediatric patients, and suitably the internal volume could range from 50 to 200 ml for neonatal patients.

91. The breathing circuit according to any one of claims 75 to 90, wherein a volume of the pressurized second gas that enters the inspirator during exhalation is in the range of the 60 to 80 percent by vol % of a tidal volume of a patient, and suitably 70 percent by vol % of a tidal volume of a patient.

92. The breathing circuit according to any one of claims 75 to 91, wherein the inspirator is sufficiently long so that the stored second gas is prevented from being discharged from the inspirator with the first gas in excess via the further positive end expiratory pressure valve.

93. The breathing circuit according to any one of claims 75 to 92, wherein the breathing circuit includes a patient delivery device.

94. The breathing circuit according to any one of claims 75 to 93, wherein the patient delivery device has an inlet connection that connects to the inspirator that supplies the fresh breathing gases, and an outlet connection that connects to the expirator.

95. The breathing circuit according to claim 94, wherein the inlet connection and the outlet connection are limbs of a Y-piece on the patient delivery device.

96. The breathing circuit according to any one of claims 75 to 95, wherein the patient delivery device is a sealed face mask.

97. The breathing circuit according to any one of claims 75 to 96, wherein the patient delivery device is a sealed cannula.

98. The breathing circuit according to any one of 75 to 97, wherein the inspirator has a movable plug with a non-return valve that provides a boundary between the stored the second gas and the first gas supplied, and the plug.

99. The breathing circuit according to any one of claims 75 to 98, wherein the first gas is pressurized air.

100. The breathing circuit according to any one of claims 75 to 99, wherein the second gas is pressurized oxygen gas.

101. The breathing circuit according to any one of claims 75 to 100, wherein the second gas is pressurized one or a combination of: oxygen gas, helium, heliox, an anaesthetic gas, or nitrous oxide.

102. A method of operating the breathing circuit according to any one of claims 75 to 101, wherein the method includes operating the breathing circuit at a positive pressure by maintaining an oversupply of the first gas into the inspirator.

103. The method according to claim 102, wherein the first gas is supplied continuously to the inspirator.

104. The method according to claim 103, wherein the first gas is supplied at a rate that is greater than or equal to peak inspiratory flow rate of a patient.

105. The method according to any one of claims 102 to 104, wherein the method includes setting the pressure at which the further positive end expiratory pressure valve vents the first gas that is in excess from the inspirator.

106. The method according to any one of claims 102 to 105, wherein the expirator has a positive end expiratory pressure valve for venting expired gas, and the inspirator has a further positive end expiratory pressure valve, and wherein the method includes selecting pressure settings of the positive end expiratory pressure valve of the expirator and the further positive end expiratory pressure valve of the inspirator so that there is differential between the pressure settings that will prevent net flow from the inspirator into the expirator other than that caused by the breathing of the patient.

107. The method according to any one of claims 102 to 106, wherein the method includes controlling the flow of second gas to the inspirator at a fixed rate depending on the requirement of the patient.

108. The method according to any one of claims 102 to 107, wherein the second gas includes enriched oxygen gas, or oxygen gas and controlling the flow rate of the oxygen gas supplied to the inspiratory member is based on level of oxygen saturation in the patient's blood.

109. The method according to claim 107 or 108, wherein controlling the flow rate of the second gas is determined independently of any one or any combination of:
i) the tidal flow of the patient;
ii) changes in tidal flow of the patient; or
iii) a flow rate at which the first gas is supplied into the inspiratory tube.

110. The method according to any one of claims 102 to 109, wherein the pressure regulation device includes a first pressure relief valve configured to control the pressure of the first gas supplied to the inspiratory member, and the method includes operating the first pressure relief valve to control the pressure at which the first is supplied to the inspiratory member.

111. The method according to any one of claims 101 to 109, wherein the pressure regulation device includes a second pressure relief valve configured to vent the first gas from the inspiratory member, and the method includes operating the second pressure relief valve to control the pressure of the inspiratory member.

112. The method according to any one of claims 102 to 111, wherein during an expiration period of the patient's breathing cycle the second gas will backfill along the inspirator which acts as a constant pressure storage volume by displacing the first gas in the inspirator out via the positive end expiratory pressure valve of the inspirator.

113. The method according to any one of claims 102 to 112, wherein during an inspiration period of the patient's breathing cycle, the fresh breathing gas drawn into the lungs from the inspirator will initially be a combination of the second gas from the source of pressurized second gas in combined with second gas that had been stored in the inspirator during the expiration period.
